(19) United States
(12) Patent Application Publication
(10) Pub. No.: US 2023/0105595 A1
(43) Pub. Date: Apr. 6, 2023

(54) PROCESS OF PREPARING 2-(PHENYLIMINO)-3-ALKYL-1,3-THIAZOLIDIN-4-ONES

(71) Applicant: Bayer Aktiengesellschaft, Leverkusen (DE)

(72) Inventors: Thomas HIMMLER, Odenthal (DE); Sergii PAZENOK, Leichlingen (DE); Julia Johanna HAHN, Duesseldorf (DE); Klaus-Ulrich SCHIFFER, Wuppertal (DE)

(21) Appl. No.: 17/800,863

(22) PCT Filed: Feb. 15, 2021
(86) PCT No.: PCT/EP2021/053603

§ 371 Date: Aug. 18, 2022

(30) Foreign Application Priority Data
Feb. 18, 2020 (EP) 20157917.4

(51) Int. Cl. C07D 277/54; C07C 323/36; C07C 335/28; C07C 335/16; C07C 319/14

(52) U.S. Cl. CPC C07D 277/54; C07C 323/36; C07C 335/28; C07C 335/16; C07C 319/14

(57) ABSTRACT

The present invention relates to a method for preparing 2-(phenylimino)-3-alkyl-1,3-thiazolidin-4-ones of the general formula (I)

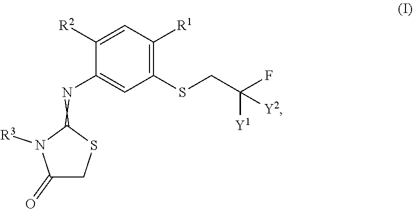

in which Y¹, Y², R¹, R² and R³ are as defined in the description.

The present invention relates to a method for preparing 2-(phenylimino)-3-alkyl-1,3-thiazolidin-4-ones of the general formula (I).

2-(Phenylimino)-3-alkyl-1,3-thiazolidin-4-ones and corresponding derivatives are of great importance in the pharmaceutical and agrochemical industry as intermediates in the production of, for example, chiral sulfoxides. Sulfoxides of this kind are used for example in crop protection as acaricides (see e.g. WO2013/092350 or WO2015/150348).

The chemical synthesis of 2-(phenylimino)-3-alkyl-1,3-thiazolidin-4-ones is known. This can be accomplished, for example, by reacting an appropriately N,N′-disubstituted thiourea of the general formula (II) with an acetic acid derivative of the general formula (III) (see e.g. WO2013/092350; EP 985670; Advances in Heterocycl. Chem. 25, (1979) 85)). There are in principle a number of methods for preparing the N,N′-disubstituted thiourea of the general formula (II). A simple and effective method consists of the reaction of an appropriately substituted aniline of the general formula (IV) with an isothiocyanate of the general formula (V) (WO2014/202510). Conversely, it is also possible to obtain in this manner the N,N-disubstituted thiourea of the general formula (II) by reacting an aryl isothiocyanate of the general formula (VI) with an amine of the general formula (VII) (JP2011/042611).

Thus, a familiar method of preparing 2-(phenylimino)-3-alkyl-1,3-thiazolidin-4-ones of the general formula (I) is characterized in that, in a first step, an aniline of the general formula (IV) is reacted with an isothiocyanate of the general formula (V), or an aryl isothiocyanate of the general formula (VI) is reacted with an amine of the general formula (VII), and the N,N′-disubstituted thiourea of the general formula (II) thereby formed is then isolated, for example by filtration. In a second step of the known method, the N,N′-disubstituted thiourea of the general formula (II) is then reacted with an acetic acid derivative of the general formula (III) in the presence of a base to form the 2-(phenylimino)-3-alkyl-1,3-thiazolidin-4-one of the general formula (I).

A disadvantage of this method is the use of isothiocyanates, namely either the alkyl isothiocyanate of the general formula (V) or the aryl isothiocyanate of the general formula (VI). Isothiocyanates can often only be prepared by laborious methods using hazardous chemicals. For instance, the preparation of isothiocyanates of the general formulae (V) and (VI) is known by reacting an amine of the general formula (VII) or an aniline of the general formula (IV) with thiophosgene (Rapid Communications in Mass Spectrometry 8 (1994) 737). In this case, the use of thiophosgene is highly disadvantageous. Thiophosgene is highly toxic; is very corrosive; has a foul odour; and is generally poorly accessible and only at high cost. Another familiar method for preparing isothiocyanates of the general formulae (V) and (VI) consists of reacting an amine of the general formula (VII) or an aniline of the general formula (IV), in the presence of a base such as triethylamine, with carbon disulfide to give dithiocarbamates of the general formula (VIII) and subsequently reacting these with reagents such as chloroformic esters (J. Org. Chem. 29 (1964) 3098), tosyl chloride (WO2012/129338), phosgene (Chem. Zentralblatt 101 (1930) Buch 1(3), 3431), sodium hypochlorite (Liebigs Ann. Chem. 585 (1954) 230), sodium chlorite (DE 960276) or hydrogen peroxide (J. Org. Chem. 62 (1997) 4539). These methods have various disadvantages such as the use of low-boiling and highly flammable carbon disulfide or the use of highly toxic phosgene. In addition, the yields for an industrial process are not high enough. The likewise known reaction of an alkyl halide with a rhodanide to give the thiocyanate and subsequent isomerization to the isothiocyanate does not work in all cases.

The method (A) known from the prior art for preparing 2-(phenylimino)-3-alkyl-1,3-thiazolidin-4-ones is shown in scheme (1), in which X, Y¹, Y², W, R¹, R² and R³ are as defined below.

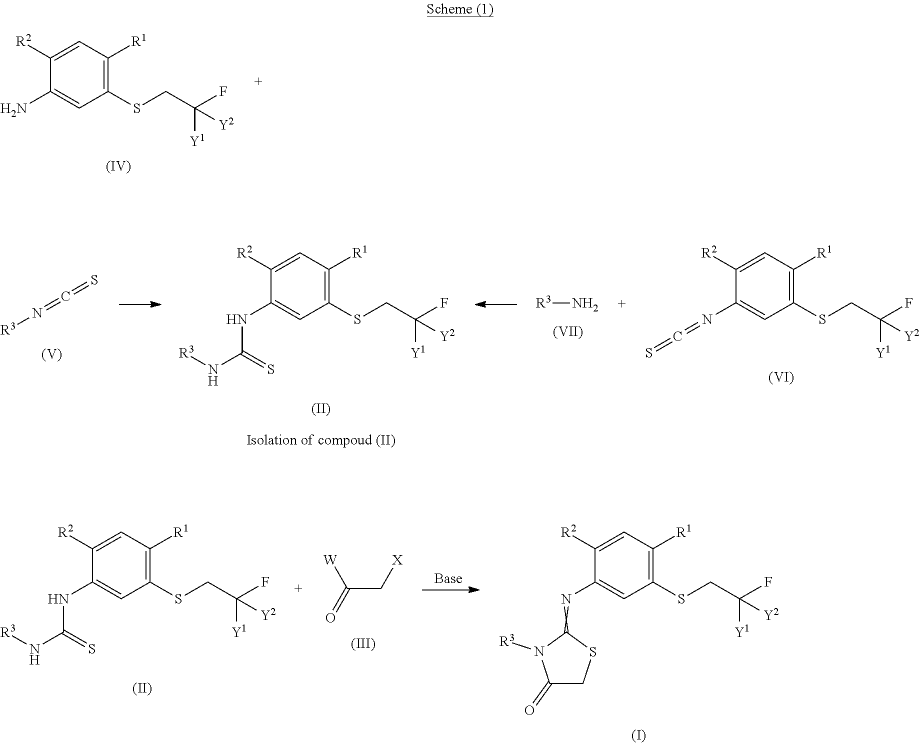

In view of the disadvantages outlined above, there is therefore an urgent need for a simplified, industrially and economically practicable method for preparing 2-(phenylimino)-3-alkyl-1,3-thiazolidin-4-ones of the general formula (I). The 2-(phenylimino)-3-alkyl-1,3-thiazolidin-4-ones obtainable with this envisaged method should preferably be afforded in high yield and high purity.

Surprisingly, it has been found that 2-(phenylimino)-3-alkyl-1,3-thiazolidin-4-ones of the general formula (I) can be prepared by reacting a 2-(phenylimino)-3H-1,3-thiazolidin-4-one of the general formula (VIII) with an alkylating agent of the general formula (IX).

The present invention accordingly provides a method (B-1) for preparing 2-(phenylimino)-3-alkyl-1,3-thiazolidin-4-ones of the general formula (I)

in which

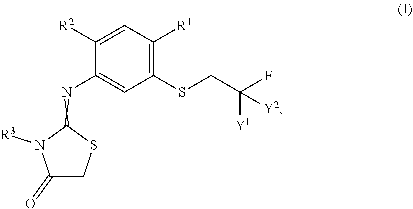

Y¹ and Y² are each independently fluorine, chlorine or hydrogen,

R¹ and R² are each independently hydrogen, (C₁-C₁₂)alkyl, (C₁-C₁₂)haloalkyl, cyano, halogen or nitro, and

R³ is optionally substituted (C₆-C₁₀)aryl, (C₁-C₁₂)alkyl or (C₁-C₁₂)haloalkyl, in which the substituents are selected from halogen, (C₁-C₆)alkyl, (C₃-C₁₀)cycloalkyl, cyano, nitro, hydroxy, (C₁-C₆)alkoxy, (C₁-C₆)haloalkyl and (C₁-C₆)haloalkoxy, in particular from fluorine, chlorine, (C₁-C₃)alkyl, (C₃-C₆)cycloalkyl, cyclopropyl, cyano, (C₁-C₃)alkoxy, (C₁-C₃)haloalkyl and (C₁-C₃)haloalkoxy,

which is characterized in that a 2-(phenylimino)-3H-1,3-thiazolidin-4-one of the general formula (VIII):

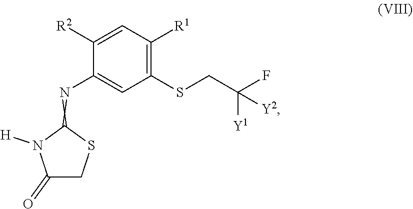

in which Y¹, Y², R¹ and R² are as defined above,

is reacted with an alkylating agent of the general formula (IX):

R³—Z (IX)

in which

R³ is as defined above,

and

Z is OSO₂F,

in the presence of a base and a solvent.

The 2-(phenylimino)-3-alkyl-1,3-thiazolidin-4-ones of the general formula (I) can be prepared by the method according to the invention with good yields and in high purity.

The compounds of the formula (I) may be present as the E- or Z-isomer or as a mixture of these isomers. This is indicated by the crossed double bond in the formula (I). In an individual embodiment of the invention, the compound is in each case in the form of the E-isomer. In another individual embodiment of the invention, the compound is in each case in the form of the Z-isomer. In another individual embodiment of the invention, the compound is in the form of a mixture of the E- and Z-isomers. In a preferred individual embodiment of the invention, the compound is in the form of the Z-isomer or a mixture of the E- and Z-isomers in which the proportion of the Z-isomer is greater than 50% and with increasing preference greater than 60%, 65%, 70%, 75%, 80%, 85%, 90%, 95%, based on the total amount of the E- and Z-isomers in the mixture.

Since the starting material of the general formula (VIII) can also react from a tautomeric form of the general formula (VIII′)

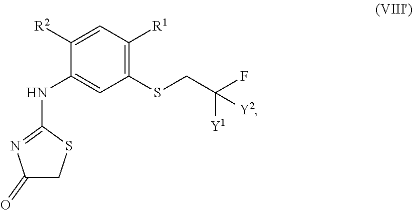

in which Y¹, Y², R¹ and R² are as defined above,

in the method according to the invention to give the compounds of the formula (I), the isomeric products of the general formula (X) (2-[{2-phenyl}(alkyl)amino]-1,3-thiazol-4(5H)-ones)

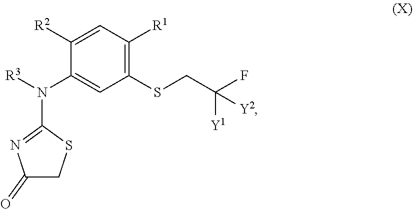

in which Y¹, Y², R¹, R² and R³ are as defined above,

may also be obtained.

The method according to the invention is also characterized in that the compounds of the formula (I) are obtained with high selectivity, i.e. in significantly higher proportions than the compounds of the general formula (X).

Preferred, particularly preferred and very particularly preferred definitions of the radicals Y¹, Y², Z, R¹, R² and R³ listed in the formulae (I), (VIII), (VIII′), (IX) and (X) mentioned above are elucidated below.

It is preferable when

Y¹ and Y² are each independently fluorine, chlorine or hydrogen,

R¹ and R² are each independently fluorine, chlorine, (C₁-C₃)alkyl or hydrogen,

R³ is (C₁-C₆)alkyl or (C₁-C₆)haloalkyl, and

Z is OSO₂F.

It is particularly preferable when

Y¹ and Y² are each independently fluorine or hydrogen,

R¹ and R² are each independently fluorine, chlorine, hydrogen or methyl,

R³ is (C₁-C₆)haloalkyl, and

Z is OSO₂F.

It is very particularly preferable when

Y¹ and Y² are fluorine,

R¹ and R² are each independently fluorine, hydrogen or methyl,

R³ is (C₁-C₆)fluoroalkyl, and

Z is OSO₂F.

It is most preferable when

Y¹ and Y² are fluorine,

R¹ is methyl,

R² is fluorine,

R³ is CH₂CF₃, and

Z is OSO₂F.

The present application also relates to a form of embodiment (B-2) of the method according to the invention, which is characterized in that the compound (IX), where Z is OSO₂F, is not used as such but is prepared in situ by reacting a compound of the general formula (XI)

R³—OH (XI)

in which

R³ has the definition previously stated,

with SO₂F₂ or SO₂ClF.

In this regard, preference is given to using SO₂F₂. Since the reaction of compound (XI) with SO₂F₂ or SO₂ClF to give compound (IX) takes place in situ, this reaction also takes place in the presence of a base and a solvent.

This form of embodiment (B-2) of the method according to the invention is preferred. It is shown in scheme (2) below.

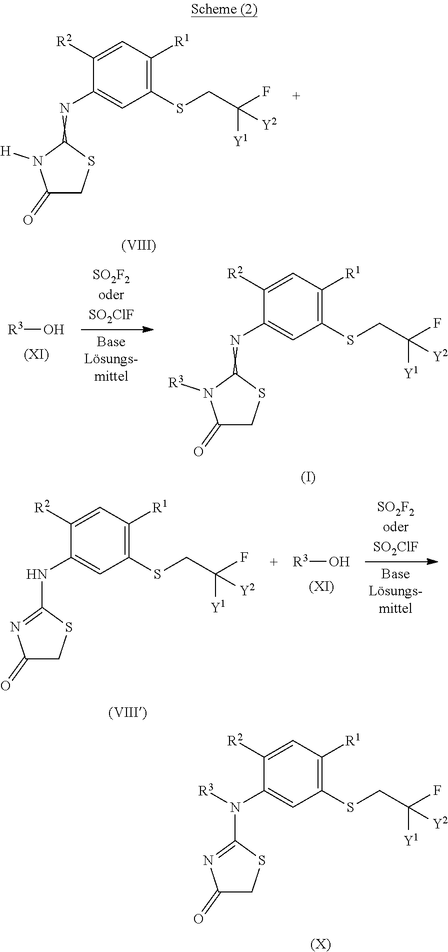

The present application likewise provides compounds of the general formula (VIII)

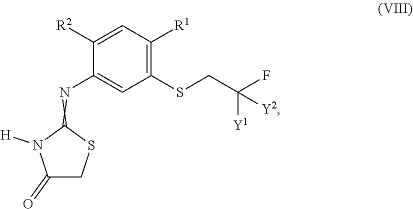

in which Y¹, Y², R¹ and R² are as defined above.

It is therefore preferable in the general formula (VIII) when

Y¹ and Y² are each independently fluorine, chlorine or hydrogen, and

R¹ and R² are each independently fluorine, chlorine, (C₁-C₃)alkyl or hydrogen.

It is therefore particularly preferable when

Y¹ and Y² are each independently fluorine or hydrogen, and

R¹ and R² are each independently fluorine, chlorine, hydrogen or methyl.

It is therefore very particularly preferable when

Y¹ and Y² are fluorine, and

R¹ and R² are each independently fluorine, hydrogen or methyl.

It is therefore most preferable when

Y¹ and Y² are fluorine,

R¹ is methyl, and

R² is fluorine.

The compounds of the general formula (VIII) can be prepared, for example, from the corresponding monoarylthioureas of the general formula (XII), in which Y¹, Y², R¹ and R² are as defined above, by reaction with a compound of the general formula (III), in which X is bromine, chlorine, OSO₂Me, OSO₂Ph, OSO₂(4-Me-Ph) or OSO₂CF₃ and W is OH or a radical O(C₁-C₆-alkyl) (scheme (3)).

This method step for preparing compounds of the general formula (VIII) can therefore be upstream of the method according to the invention, particularly the forms of embodiment B-1 and B-2. Consequently, this represents a further separate embodiment of the method according to the invention (forms of embodiment B-1.1 and B-2.1).

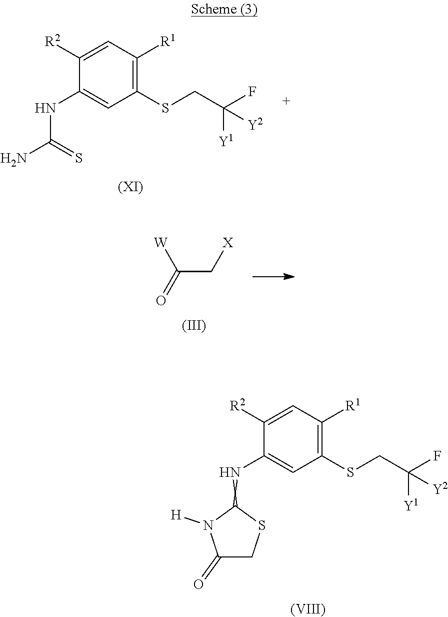

It is preferable when X is bromine or chlorine and W is a radical O(C₁-C₆-alkyl). It is very particularly preferable when X is bromine or chlorine and W is a radical OCH₃ or OC₂H₅. It is most preferable when X is bromine or chlorine and W is a radical OCH₃.

The present application therefore further provides compounds of the general formula (XII)

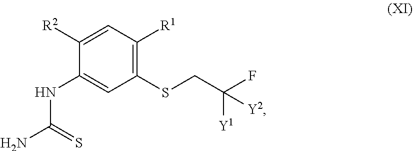

in which Y¹, Y², R¹ and R² are as defined above.

It is therefore preferable in the general formula (XII) when

Y¹ and Y² are each independently fluorine, chlorine or hydrogen, and

R¹ and R² are each independently fluorine, chlorine, (C₁-C₃)alkyl or hydrogen.

It is therefore particularly preferable when

Y¹ and Y² are each independently fluorine or hydrogen, and

R¹ and R² are each independently fluorine, chlorine, hydrogen or methyl.

It is therefore very particularly preferable when

Y¹ and Y² are fluorine, and

R¹ and R² are each independently fluorine, hydrogen or methyl.

It is therefore most preferable when

Y¹ and Y² are fluorine,

R¹ is methyl, and

R² is fluorine.

Monoarylthioureas of the general formula (XII) can be prepared by various methods. A preferred method consists in that an aniline of the general formula (IV)

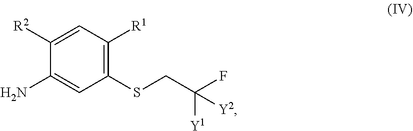

in which Y¹, Y², R¹ and R² are as defined above,

is reacted with an alkoxycarbonyl isothiocyanate of the general formula (XIII)

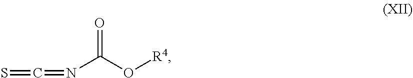

in which R⁴ is methyl, ethyl or isopropyl,

to give an alkyl (phenylcarbamothioyl)carbamate of the general formula (XIV)

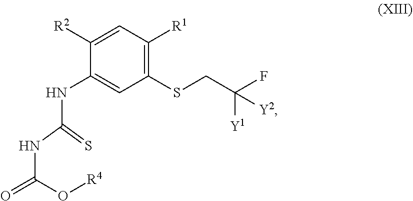

in which Y¹, Y², R¹, R² and R⁴ are as defined above,

and the compound of the general formula (XIV) is then saponified and decarboxylated under acidic or alkaline conditions to give the monoarylthiourea of the general formula (XII) (scheme (4)). Saponification and decarboxylation are well-known in this regard to those skilled in the art and described many times in the prior art.

This method step for preparing compounds of the general formula (XII) can therefore be upstream of the method according to the invention, particularly the forms of embodiment B-1.1 and B-2.1. Consequently, this represents a further separate embodiment of the method according to the invention (forms of embodiment B-1.1.1 and B-2.1.1).

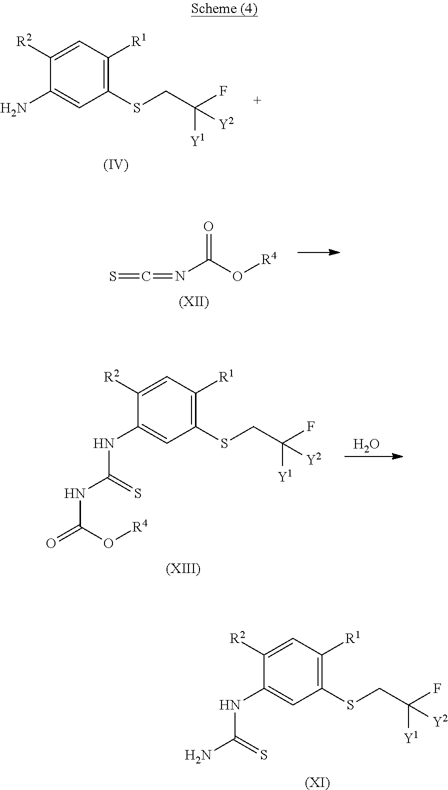

The present application therefore also provides alkyl (phenylcarbamothioyl)carbamates of the general formula (XIV):

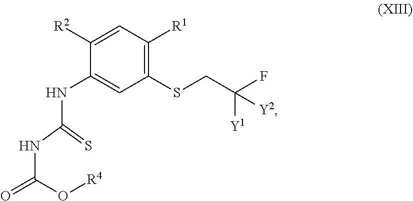

in which Y¹, Y², R¹, R² and R⁴ are as defined above.

It is therefore preferable in the general formula (XIV) when

Y¹ and Y² are each independently fluorine, chlorine or hydrogen,

R¹ and R² are each independently fluorine, chlorine, (C₁-C₃)alkyl or hydrogen, and

R⁴ is methyl, ethyl or isopropyl.

It is therefore particularly preferable when

Y¹ and Y² are each independently fluorine or hydrogen,

R¹ and R² are each independently fluorine, chlorine, hydrogen or methyl, and

R⁴ is methyl or ethyl.

It is therefore very particularly preferable when

Y¹ and Y² are fluorine,

R¹ and R² are each independently fluorine, hydrogen or methyl, and

R⁴ is methyl or ethyl.

It is therefore most preferable when

Y¹ and Y² are fluorine,

R¹ is methyl,

R² is fluorine, and

R⁴ is methyl or ethyl.

A further possibility for preparing compounds of the general formula (VIII) consists of reacting 2-halo-N-(phenyl)acetamides of the general formula (XV)

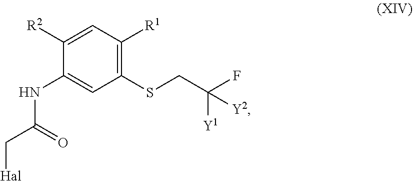

in which Y¹, Y², R¹ and R² are as defined above

and

Hal is chlorine or bromine,

with an alkali metal or ammonium rhodanide of the general formula (XVI):

MSCN (XVI),

in which M is Li, Na, K or NH₄.

This reaction is shown in Scheme 5. This method step for preparing compounds of the general formula (VIII) can therefore also be upstream of the method according to the invention, particularly the forms of embodiment B-1 and B-2. Consequently, this represents a further separate embodiment of the method according to the invention (forms of embodiment B-1.2 and B-2.2).

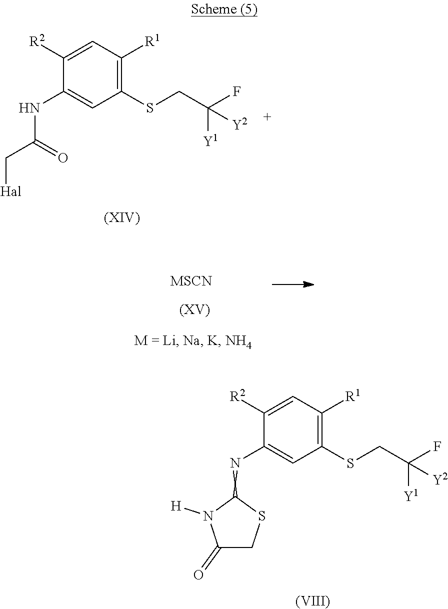

The present application therefore also provides 2-halo-N-(phenyl)acetamides of the general formula (XV)

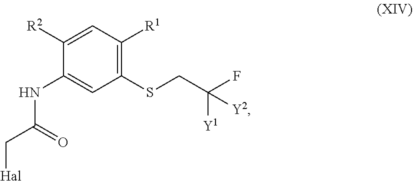

in which Y¹, Y², R¹, R² and Hal are as defined above.

It is therefore preferable in the general formula (XV) when

Y¹ and Y² are each independently fluorine, chlorine or hydrogen,

R¹ and R² are each independently fluorine, chlorine, (C₁-C₃)alkyl or hydrogen, and

Hal is bromine or chlorine.

It is therefore particularly preferable when

Y¹ and Y² are each independently fluorine or hydrogen,

R¹ and R² are each independently fluorine, chlorine, hydrogen or methyl, and

Hal is bromine or chlorine.

It is therefore very particularly preferable when

Y¹ and Y² are fluorine,

R¹ and R² are each independently fluorine, hydrogen or methyl, and

Hal is chlorine.

It is therefore most preferable when

Y¹ and Y² are fluorine,

R¹ is methyl,

R² is fluorine and

Hal is chlorine.

The 2-halo-N-(phenyl)acetamides of the general formula (XV) can be obtained by reacting anilines of the general formula (IV) (as specified above) with a haloacetyl halide of the general formula (XVII):

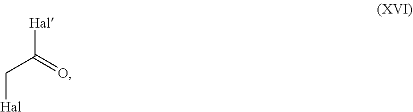

in which Hal and Hal′ are each independently chlorine or bromine, especially preferably chlorine.

This method step for preparing compounds of the general formula (XV) can therefore be upstream of the method according to the invention, particularly the forms of embodiment B-1.2 and B-2.2. Consequently, this represents a further separate embodiment of the method according to the invention (forms of embodiment B-1.2.1 and B-2.2.1).

The method according to the invention is shown in its entirety in Scheme 6.

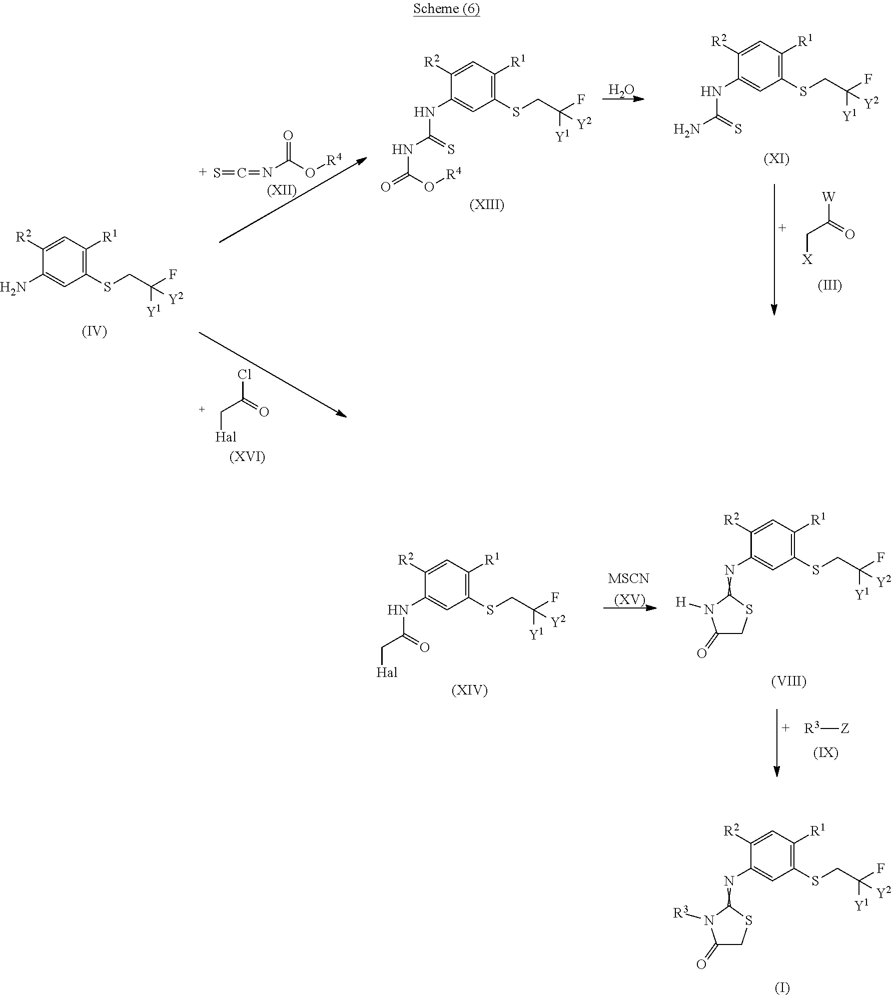

All forms of embodiment of methods according to the invention enable the 2-(phenylimino)-3-alkyl-1,3-thiazolidin-4-ones of the general formula (I) to be prepared in good yields and in high purity.

### General Definitions

In the context of the present invention, the term halogens (Hal) encompasses, unless otherwise defined at the relevant position, those elements selected from the group consisting of fluorine, chlorine, bromine and iodine, preference being given to using fluorine, chlorine and bromine, and particular preference to using fluorine and chlorine.

Optionally substituted groups may be singly or multiply substituted; if multiply substituted, the substituents may be identical or different. Unless otherwise stated at the relevant position, substituents are selected from halogen, (C₁-C₆)alkyl, (C₃-C₁₀)cycloalkyl, cyano, nitro, hydroxy, (C₁-C₆)alkoxy, (C₁-C₆)haloalkyl and (C₁-C₆)haloalkoxy, in particular from fluorine, chlorine, (C₁-C₃)alkyl, (C₃-C₆)cycloalkyl, cyclopropyl, cyano, (C₁-C₃)alkoxy, (C₁-C₃)haloalkyl and (C₁-C₃)haloalkoxy.

Alkyl groups substituted by one or more halogen atoms (Hal) are, for example, selected from trifluoromethyl (CF₃), difluoromethyl (CHF₂), CF₃CH₂, ClCH₂ or CF₃CCl₂.

Alkyl groups in the context of the present invention are, unless otherwise defined, linear, branched or cyclic saturated hydrocarbon groups.

The definition C₁-C₁₂-alkyl encompasses the widest range defined herein for an alkyl group. Specifically, this definition encompasses, for example, methyl, ethyl, n-propyl, isopropyl, n-butyl, isobutyl, sec-butyl and t-butyl, n-pentyl, n-hexyl, 1,3-dimethylbutyl, 3,3-dimethylbutyl, n-heptyl, n-nonyl, n-decyl, n-undecyl, n-dodecyl.

Aryl groups in the context of the present invention are, unless otherwise defined, aromatic hydrocarbon groups, which may comprise one, two or more heteroatoms (selected from O, N, P and S).

Specifically, this definition encompasses, for example, cyclopentadienyl, phenyl, cycloheptatrienyl, cyclooctatetraenyl, naphthyl and anthracenyl; 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; 1-pyrrolyl, 1-pyrazolyl, 1,2,4-triazol-1-yl, 1-imidazolyl, 1,2,3-triazol-1-yl, 1,3,4-triazol-1-yl; 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

The reaction of the 2-(phenylimino)-3H-1,3-thiazolidin-4-one of the general formula (VIII) to give the compound of the formula (I) is carried out according to the invention in the presence of a solvent. Suitable solvents in the method according to the invention are in particular the following: dichloromethane, acetonitrile, propionitrile, butyronitrile, ethyl acetate, butyl acetate, toluene, chlorobenzene, N,N-dimethylformamide, N,N-dimethylacetamide, N-methylpyrrolidinone, dimethyl sulfoxide and sulfolane. Mixtures of said solvents may also be used.

Preferred solvents are dichloromethane, acetonitrile, butyronitrile, N,N-dimethylformamide, N,N-dimethylacetamide, N-methylpyrrolidinone, dimethyl sulfoxide, sulfolane or mixtures of said solvents.

Particularly preferred solvents are acetonitrile, N,N-dimethylacetamide, N-methylpyrrolidinone, dimethyl sulfoxide or mixtures of said solvents.

In the form of embodiment (B-1) and the further embodiments of methods according to the invention including this form of embodiment, the alkylating agent R³—Z of the general formula (IX) is preferably used at a molar ratio from 0.9:1 to 2:1, based on the 2-(phenylimino)-3H-1,3-thiazolidin-4-one of the general formula (VIII). Further preference is given to molar ratios from 0.95:1 to 2.5:1, again in each case based on the 2-(phenylimino)-3H-1,3-thiazolidin-4-one of the general formula (VIII).

If the alkylating agent R³—Z of the general formula (IX) in the form of embodiment (B-2), and the further embodiments of methods according to the invention including this form of embodiment, is prepared from an alcohol R³—OH of the general formula (XI) and SO₂F₂ or SO₂ClF in situ, then the alcohol R³—OH is preferably used in a molar ratio from 1:1 to 4:1, based on the 2-(phenylimino)-3H-1,3-thiazolidin-4-one of the general formula (VIII).

The reagent SO₂F₂ or SO₂ClF required to prepare the alkylating agent R³—Z of the general formula (IX) in the form of embodiment (B-2), and the further embodiments of methods according to the invention including this form of embodiment, is preferably used at a molar ratio from 1:1 to 4:1, preferably from 1.1:1 to 2.5:1, based in each case on the 2-(phenylimino)-3H-1,3-thiazolidin-4-one of the general formula (VIII).

The method according to the invention is carried out in the presence of a base.

The base used in the method according to the invention may be organic and inorganic bases. Organic bases include, for example, trimethylamine, triethylamine, tributylamine, ethyldiisopropylamine, pyridine, 2-methylpyridine, 2,3-dimethylpyridine, 2,5-dimethylpyridine, 2,6-dimethylpyridine, 2-methyl-5-ethylpyridine, quinoline, potassium methoxide, potassium ethoxide, potassium tert-butoxide, sodium methoxide, sodium ethoxide, sodium tert-butoxide, potassium acetate and sodium acetate. Inorganic bases include, for example, lithium hydroxide, potassium hydroxide, sodium hydroxide, potassium hydrogen carbonate, sodium hydrogen carbonate, potassium carbonate, sodium carbonate, caesium carbonate, calcium carbonate and magnesium carbonate. Preference is given to triethylamine, tributylamine, ethyldiisopropylamine, 2-methyl-5-ethylpyridine, sodium methoxide, potassium hydrogencarbonate, sodium hydrogencarbonate, potassium carbonate and sodium carbonate. Particular preference is given to triethylamine, tributylamine, sodium hydrogencarbonate, potassium hydrogencarbonate, potassium carbonate, sodium carbonate and sodium methoxide.

In the form of embodiment (B-1) and the further embodiments of methods according to the invention including this form of embodiment, the base is preferably used at a molar ratio from 0.9:1 to 4:1, based on the 2-(phenylimino)-3H-1,3-thiazolidin-4-one of the general formula (VIII). Further preference is given to molar ratios from 1:1 to 2:1, again in each case based on the 2-(phenylimino)-3H-1,3-thiazolidin-4-one of the general formula (VIII).

In the form of embodiment (B-2) and the further embodiments of methods according to the invention including this form of embodiment, the base is preferably used at a molar ratio from 1:1 to 4:1, based on the 2-(phenylimino)-3H-1,3-thiazolidin-4-one of the general formula (VIII)t. Further preference is given to molar ratios from 1.5:1 to 3:1, again in each case based on the 2-(phenylimino)-3H-1,3-thiazolidin-4-one of the general formula (VII).

All forms of embodiment of methods according to the invention are generally carried out at a temperature between −20° C. and 150° C., preferably between 0° C. and 120° C., most preferably between 5° C. and 80° C.

The reaction is typically carried out at standard pressure, but may also be carried out at elevated or reduced pressure.

The desired compounds of the formula (I) may be isolated for example by subsequent filtration or extraction. Such processes are well-known to those skilled in the art.

The present invention is elucidated in detail by the examples that follow, although the examples should not be interpreted in such a manner that they restrict the invention.

### PREPARATION EXAMPLES

### Example 1: Synthesis of 2-chloro-N-{2-fluoro-4-methyl-5-[(2,2,2-trifluoroethyl)sulfanyl]phenyl}acetamide

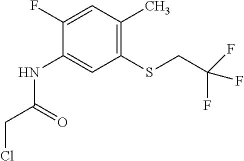 [SMILES: Cc1cc(F)c(NC(=O)CCl)cc1SCC(F)(F)F]

To a solution of 11.96 g [50 mmol] of 2-fluoro-4-methyl-5-[(2,2,2-trifluoroethyl)sulfanyl]aniline and 10.12 g [100 mmol] of triethylamine in 100 ml of methylene chloride were added dropwise 6.78 g [60 mmol] of chloroacetyl chloride at 0-5° C. The mixture was stirred for 1 hour at 0-5° C. and then overnight at 20° C. The reaction mixture was stirred with 150 ml of water. The organic phase was separated off, the aqueous phase extracted with 50 ml of methylene chloride, the combined organic phases washed twice with 50 ml of 15% hydrochloric acid and then with 50 ml of water, dried over sodium sulfate and concentrated under reduced pressure. This gave 15 g of brownish solid which, according to GC (gas chromatography), had a purity of 96.5% (a/a), which resulted in a yield of 92.9% of theory.

Melting point: 128° C.

GC/MS: m/e=315 (M⁺, 1 Cl, 33%), 239 (M*-76, 43%), 156 (100%).

¹H-NMR (600 MHz, d₆-DMSO): δ=2.44 (s, 3H), 3.87 (q, 2H), 4.4 (s, 2H), 7.32 (d, 1H), 8.12 (d, 1H), 10.17 (s, 1H) ppm.

¹⁹F-NMR (565 MHz, d₆-DMSO): δ=−64.3 (t, 3F), −124.3 (dd, 1F) ppm.

### Example 2: Synthesis of methyl ({2-fluoro-4-methyl-5-[(2,2,2-trifluoroethyl)sulfanylphenyl}carbamothioyl)carbamate

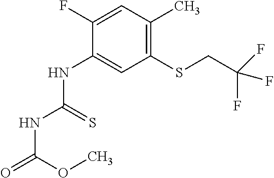 [SMILES: COC(=O)NC(=S)Nc1cc(SCC(F)(F)F)c(C)cc1F]

Step 1 (preparation of methoxycarbonyl isothiocyanate): To 56.75 g [0.7 mol] of sodium thiocyanate in 300 ml of toluene was added 0.4 g of pyridine and 0.9 g of water at 30° C. Subsequently, 56.7 g [0.6 mol] of methyl chloroformate were added over 20 minutes. The mixture was stirred at 30° C. for 2 hours, cooled to 20° C. and the sodium chloride filtered off. The filtrate was used in step 2.

Step 2 (preparation of the title compound): The filtrate from step 1 was initially charged and a solution of 119.6 g [0.5 mol] of 2-fluoro-4-methyl-5-[(2,2,2-trifluoroethyl)sulfanyl]aniline in 100 ml of toluene was added at 30° C. After completion of the addition, the mixture was heated to 80° C. and stirred for 90 minutes at this temperature. The reaction mixture was then cooled to 0° C., the precipitated solid filtered off, washed with 250 ml of pentane and dried. In this manner, 165.5 g of white solid was obtained which, according to quantitative ¹H-NMR, had a content of 98.1% (w/w). This therefore corresponded to a yield of 91.1% of theory.

Melting point: 153-154° C.

¹H-NMR (600 MHz, d₆-DMSO): δ=2.40 (s, 3H), 3.76 (s, 2H), 3.86 (q, 2H), 7.28 (d, 1H), 8.05 (d, 1H), 11.36 (s, 1H), 11.55 (s, 1H) ppm. ¹⁹F-NMR (565 MHz, d₆-DMSO): δ=−64.4 (t, 3F), −123.3 (dd, 1F) ppm.

### Example 3: Synthesis of ethyl ({2-fluoro-4-methyl-5-[(2,2,2-trifluoroethyl)sulfanyl]phenyl}carbamothioyl)carbamate

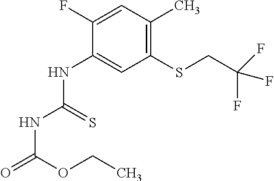 [SMILES: CCOC(=O)NC(=S)Nc1cc(SCC(F)(F)F)c(C)cc1F]

Step 1 (preparation of ethoxycarbonyl isothiocyanate): To 5.35 g [0.066 mol] of sodium thiocyanate in 50 ml of acetone are added 6.51 g [0.06 mol] of ethyl chloroformate over 5 minutes. The mixture was stirred for 15 minutes under reflux, cooled to 20° C. and the sodium chloride filtered off. The filtrate was used in step 2.

Step 2 (preparation of the title compound): The filtrate from step 1 was initially charged and, at 20° C. initially without cooling, a solution of 11.96 g [0.05 mol] of 2-fluoro-4-methyl-5-[(2,2,2-trifluoroethyl)sulfanyl]aniline in 20 ml of acetone was added. After completion of the addition, the mixture was heated for 1 hour under reflux. The reaction mixture was then cooled to 20° C., added to 370 ml of water, the precipitated solid was filtered off and dried. In this manner, 19.25 g of white solid was obtained which, according to HPLC analysis, had a purity of 92.6% (a/a). This therefore corresponded to a yield of 96% of theory.

Melting point: 126° C.

LC/MS: m/e=371 (MH⁺).

¹H-NMR (600 MHz, d₆-DMSO): δ=1.26 (t, 3H), 2.4 (s, 3H), 3.86 (q, 2H), 4.22 (q, 2H), 7.28 (d, 1H), 8.05 (d, 1H), 11.4 (s, 1H), 11.5 (s, 1H) ppm.

### Example 4: Synthesis of 1-{2-fluoro-4-methyl-5-[(2,2,2-trifluoroethyl)sulfanyl]phenyl}thiourea

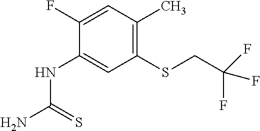 [SMILES: Cc1cc(F)c(NC(N)=S)cc1SCC(F)(F)F]

To a mixture of 893 ml of 1 N aqueous sodium hydroxide solution and 530 ml of ethanol charged in a 2 litre reactor were metered in 169.6 g [0.458 mol] of ethyl ({2-fluoro-4-methyl-5-[(2,2,2-trifluoroethyl)sulfanyl]phenyl}carbamothioyl)carbamate over ca. 10 minutes. The mixture was heated over 30 minutes to 50° C. and stirred at this temperature for 17 hours. The reaction mixture was cooled and, at about 40° C., emptied out of the reactor. At 20° C., the pH was adjusted to 6-8 with semi-concentrated hydrochloric acid. The precipitated solids were filtered off under suction, washed with water and dried. This gave 130.38 g of the title compound which, according to quantitative ¹⁹F-NMR, had a content of 94.7% (w/w). This therefore corresponded to a yield of 90.4% of theory.

Melting point: 120-122° C.

LC/MS: m/e=299 (MH⁺).

¹H-NMR (600 MHz, d₆-DMSO): δ=2.37 (s, 3H), 3.85 (q, 2H), 4.22 (q, 2H), 7.22 (d, 1H), 7.86 (d, 1H), 9.38 (s, 1H) ppm.

¹⁹F-NMR (565 MHz, d₆-DMSO): δ=−64.8 (t, 3 F), −123.5 (dd, 1F) ppm.

### Example 5: Synthesis of (2Z)-2-({2-fluoro-4-methyl-5-[(2,2,2-trifluoroethyl)sulfanyl]phenyl}imino)-1,3-thiazolidin-4-one

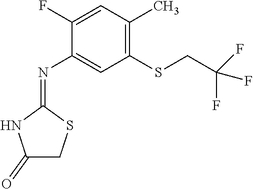 [SMILES: Cc1cc(F)c(/N=C2/NC(=O)CS2)cc1SCC(F)(F)F]

In 75 ml of acetonitrile were initially charged 14.92 g [50 mmol] of 1-{2-fluoro-4-methyl-5-[(2,2,2-trifluoroethyl)sulfanyl]phenyl}thiourea and 5.33 g [65 mmol] of sodium acetate. At 20 to 25° C., 9.18 g [55 mmol] of ethyl bromoacetate were added dropwise. The reaction mixture was stirred at 20° C. for 20 hours. The acetonitrile was then mostly distilled off under reduced pressure and 100 ml of water was added to the residue. The mixture was stirred with 100 ml of methylene chloride. The precipitated solid was filtered off and dried. In this manner 2.60 g of solid were obtained which, according to HPLC analysis, had a purity of 99.3% (a/a), which corresponded to a yield of 15.3% of theory. The methylene chloride phase was separated off, dried and concentrated. This gave 12.72 g of the title compound at a purity of 97.6% (a/a), which corresponded to a yield of 73.4% of theory.

Melting point: 128° C.

LC/MS: m/e=339 (MH⁺).

¹H-NMR (600 MHz, d₆-DMSO): δ=2.36 (s, 3H), 3.87 (q, 2H), 4.03 (s, 2H), 7.33 (m, 2H), 11.98 (s, 1H) ppm.

### Example 6: Synthesis of (2Z)-2-({2-fluoro-4-methyl-5-[(2,2,2-trifluoroethyl)sulfanyl]phenyl}imino)-1,3-thiazolidin-4-one

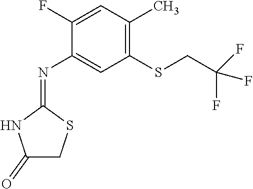 [SMILES: Cc1cc(F)c(/N=C2/NC(=O)CS2)cc1SCC(F)(F)F]

A mixture of 3.16 g [10 mmol] of 2-chloro-N-{2-fluoro-4-methyl-5-[(2,2,2-trifluoroethyl)sulfanyl]phenyl}acetamide and 1.14 g [15 mmol] of ammonium rhodanide in 25 ml of ethanol was heated under reflux for 15 hours. Subsequently, 50 ml of water and 50 ml of methylene chloride were added to the reaction mixture at room temperature. The organic phase was separated off, the aqueous phase extracted again with 50 ml of methylene chloride, the organic phases combined, washed with 50 ml of water, dried over sodium sulfate and concentrated under reduced pressure. This gave 3.33 g of product at a purity of 70.8% (a/a) according to GC/MS analysis (70% of theory).

### Example 7: Synthesis of (2Z)-2-({2-fluoro-4-methyl-5-[(2,2,2-trifluoroethyl)sulfanyl]phenyl}imino)-3-(2,2,2-trifluoroethyl)-1,3-thiazolidin-4-one (compound A) and 2-[{2-fluoro-4-methyl-5-[(2,2,2-trifluoroethyl)sulfanyl]phenyl}(2,2,2-trifluoroethyl)amino]-1,3-thiazol-4(5H)-one (compound B)

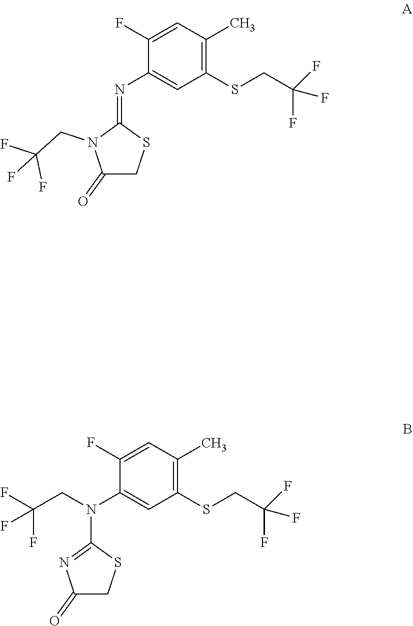 [SMILES: Cc1cc(F)c(/N=C2\SCC(=O)N2CC(F)(F)F)cc1SCC(F)(F)F ; Cc1cc(F)c(N(CC(F)(F)F)C2=NC(=O)CS2)cc1SCC(F)(F)F]

A mixture of 9.81 g [29 mmol] of (2Z)-2-({2-fluoro-4-methyl-5-[(2,2,2-trifluoroethyl)sulfanyl]phenyl}imino)-1,3-thiazolidin-4-one, 5.8 g {58 mmol] of 2,2,2-trifluoroethanol and 15 g [116 mmol] of ethyldiisopropylamine (Hünig base) in 200 ml of N,N-dimethylacetamide (DMAC) was stirred at 20° C. for 30 minutes. Then, at 20° C., 9.7 g [95 mmol] of sulfuryl fluoride (SO₂F₂) was introduced and the mixture stirred for 2 hours until the starting material had been virtually completely reacted according to HPLC monitoring. The reaction mixture was concentrated under reduced pressure, the residue taken up in tert-butyl methyl ether (MTBE), extracted twice with water, dried and evaporated. This gave 12.5 g of a thick oil. Analysis by quantitative ¹⁹F-NMR showed a content of 77.8% (w/w) of (2Z)-2-({2-fluoro-4-methyl-5-[(2,2,2-trifluoroethyl)sulfanyl]phenyl}imino)-3-(2,2,2-trifluoroethyl)-1,3-thiazolidin-4-one (corresponding to a yield of 79.8% of theory) and 12.2% (w/w) of 2-[{2-fluoro-4-methyl-5-[(2,2,2-trifluoroethyl)sulfanyl]phenyl}(2,2,2-trifluoroethyl)amino]-1,3-thiazol-4(5H)-one (corresponding to a yield of 12.5% of theory). The ratio of (2Z)-2-({2-fluoro-4-methyl-5-[(2,2,2-trifluoroethyl)sulfanyl]phenyl}imino)-3-(2,2,2-trifluoroethyl)-1,3-thiazolidin-4-one to 2-[{2-fluoro-4-methyl-5-[(2,2,2-trifluoroethyl)sulfanyl]phenyl}(2,2,2-trifluoro-ethyl)amino]-1,3-thiazol-4(5H)-one was thus 86.4:13.6.

### Example 8: Synthesis of (2Z)-2-({2-fluoro-4-methyl-5-[(2,2,2-trifluoroethyl)sulfanyl]phenyl}imino-3-(2,2,2-trifluoroethyl)-1,3-thiazolidin-4-one in CH₂Cl₂

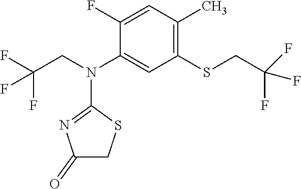 [SMILES: Cc1cc(F)c(N(CC(F)(F)F)C2=NC(=O)CS2)cc1SCC(F)(F)F]

A mixture of 0.98 g [2.9 mmol] of (2Z)-2-({2-fluoro-4-methyl-5-[(2,2,2-trifluoroethyl)sulfanyl]phenyl}imino)-1,3-thiazolidin-4-one, 0.58 g {5.8 mmol] of 2,2,2-trifluoroethanol and 1.5 g [11.6 mmol] of ethyldiisopropylamine (Hünig base) in 20 ml of dichloromethane was stirred at 20° C. for 30 minutes. Then, at 20° C., 0.8 g [7.8 mmol] of sulfuryl fluoride (SO₂F₂) was introduced over 4 hours and the mixture further stirred at RT for 12 hours. HPLC monitoring showed that the starting material had been virtually completely reacted. The reaction mixture was concentrated under reduced pressure, the residue taken up in tert-butyl methyl ether (MTBE), extracted twice with water, dried and evaporated. This gave 1.2 g of a thick oil. According to quantitative NMR, the ratio of (2Z)-2-({2-fluoro-4-methyl-5-[(2,2,2-trifluoroethyl)sulfanyl]phenyl}imino)-3-(2,2,2-trifluoroethyl)-1,3-thiazolidin-4-one to 2-[{2-fluoro-4-methyl-5-[(2,2,2-trifluoroethyl)sulfanyl]phenyl}(2,2,2-trifluoroethyl)amino]-1,3-thiazol-4(5H)-one was 64:27. Yield of (2Z)-2-({2-fluoro-4-methyl-5-[(2,2,2-trifluoroethyl)sulfanyl]phenyl}imino)-3-(2,2,2-trifluoroethyl)-1,3-thiazolidin-4-one was 58% and of 2-[{2-fluoro-4-methyl-5-[(2,2,2-trifluoroethyl)sulfanyl]phenyl}(2,2,2-trifluoroethyl)amino]-1,3-thiazol-4(5H)-one was 22%.

### Example 9: Synthesis of (2Z)-2-({2-fluoro-4-methyl-5-[(2,2,2-trifluoroethyl)sulfanyl]phenyl}imino)-3-(2,2,2-trifluoroethyl)-1,3-thiazolidin-4-one in DMF

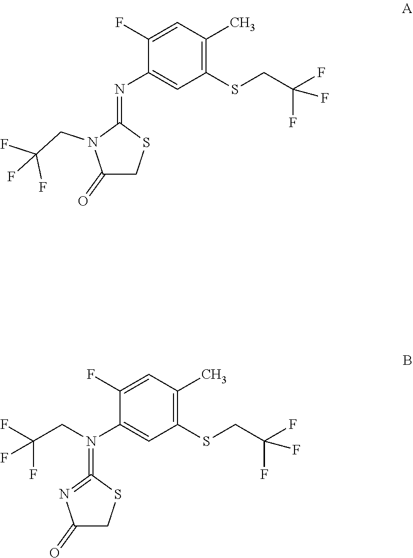

A mixture of log [29.5 mmol] of (2Z)-2-({2-fluoro-4-methyl-5-[(2,2,2-trifluoroethyl)sulfanyl]phenyl}imino)-1,3-thiazolidin-4-one, 5.9 g {59 mmol] of 2,2,2-trifluoroethanol and 11.4 g [88.4 mmol] of ethyldiisopropylamine (Hünig base) in 150 ml DMF was stirred at 20° C. for 30 minutes. Then, at 20° C., 9 g [88.5 mmol] of sulfuryl fluoride (SO₂F₂) was introduced over 4 hours and the mixture further stirred at RT for 10 hours. The reaction mixture was concentrated under reduced pressure, the residue taken up in tert-butyl methyl ether (MTBE), extracted twice with water, dried and evaporated. This gave 12.4 g of a thick oil. Analysis by quantitative ¹⁹F-NMR showed a content of 77% (w/w) of (2Z)-2-({2-fluoro-4-methyl-5-[(2,2,2-trifluoroethyl)sulfanyl]phenyl}imino)-3-(2,2,2-trifluoroethyl)-1,3-thiazolidin-4-one (corresponding to a yield of 77.0% of theory) and 13% (w/w) of 2-[{2-fluoro-4-methyl-5-[(2,2,2-trifluoroethyl)sulfanyl]phenyl}(2,2,2-trifluoroethyl)amino]-1,3-thiazol-4(5H)-one (corresponding to a yield of 13% of theory).

### Example 10: Synthesis of (2Z)-2-({2-fluoro-4-methyl-5-[(2,2,2-trifluoroethyl)sulfanyl]phenyl}imino)-3-(2,2,2-trifluoroethyl)-1,3-thiazolidin-4-one

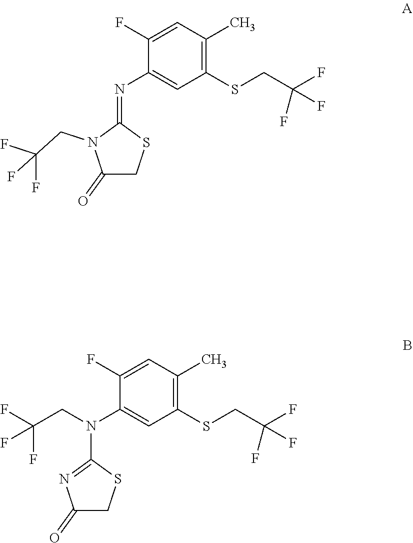 [SMILES: Cc1cc(F)c(/N=C2\SCC(=O)N2CC(F)(F)F)cc1SCC(F)(F)F ; Cc1cc(F)c(N(CC(F)(F)F)C2=NC(=O)CS2)cc1SCC(F)(F)F]

A mixture of 9.81 g [29 mmol] of (2Z)-2-({2-fluoro-4-methyl-5-[(2,2,2-trifluoroethyl)sulfanyl]phenyl}imino)-1,3-thiazolidin-4-one, 5.8 g {58 mmol] of 2,2,2-trifluoroethanol and 8.7 g [87 mmol] of K₂CO₃, pot ash, in 200 ml of N,N-dimethylacetamide (DMAC) was stirred at 20° C. for 30 minutes. Then, at 20° C., 9.7 g [95 mmol] of sulfuryl fluoride (SO₂F₂) was introduced and the mixture stirred for 2 hours until the starting material had been virtually completely reacted according to HPLC monitoring. The reaction mixture was concentrated under reduced pressure, water was added and the residue taken up in tert-butyl methyl ether (MTBE), extracted twice with water, dried and evaporated. This gave 12.4 g of a thick oil. Analysis by quantitative ¹⁹F-NMR showed a content of 78% (w/w) of (2Z)-2-({2-fluoro-4-methyl-5-[(2,2,2-trifluoroethyl)sulfanyl]phenyl}imino)-3-(2,2,2-trifluoroethyl)-1,3-thiazolidin-4-one (corresponding to a yield of 79.4% of theory) and 12% (w/w) of 2-[{2-fluoro-4-methyl-5-[(2,2,2-trifluoroethyl)sulfanyl]phenyl}(2,2,2-trifluoroethyl)amino]-1,3-thiazol-4(5H)-one (corresponding to a yield of 12.2% of theory).

### Example 11: Synthesis of (2Z)-2-({2-fluoro-4-methyl-5-[(2,2,2-trifluoroethyl)sulfanyl]phenyl}imino)-3-(2,2,2-trifluoroethyl)-1,3-thiazolidin-4-one in toluene

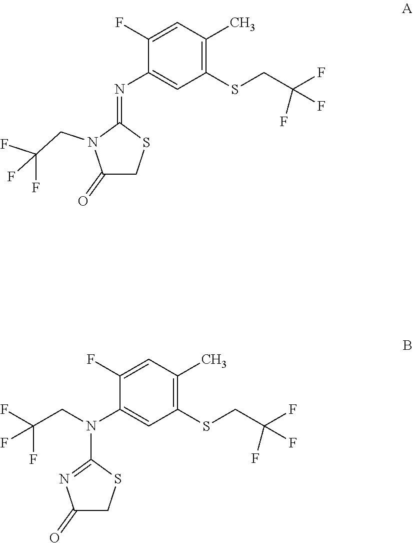 [SMILES: Cc1cc(F)c(/N=C2\SCC(=O)N2CC(F)(F)F)cc1SCC(F)(F)F ; Cc1cc(F)c(N(CC(F)(F)F)C2=NC(=O)CS2)cc1SCC(F)(F)F]

A mixture of log [29.5 mmol] of (2Z)-2-({2-fluoro-4-methyl-5-[(2,2,2-trifluoroethyl)sulfanyl]phenyl}imino)-1,3-thiazolidin-4-one, 5.9 g {59 mmol] of 2,2,2-trifluoroethanol and 11.4 g [88.3 mmol] of ethyldiisopropylamine (Hünig base) in 140 ml of toluene was stirred at 20° C. for 30 minutes. Then, at 20° C., 9 g [88.5 mmol] of sulfuryl fluoride (SO₂F₂) were introduced over 6 hours and the mixture stirred at 20° C. for 20 hours. The reaction mixture was washed with water and the toluene concentrated under reduced pressure. The residue of 12.5 g was analyzed by NMR.

⁹F-NMR showed a content of 31% (w/w) of (2Z)-2-({2-fluoro-4-methyl-5-[(2,2,2-trifluoroethyl)sulfanyl]phenyl}imino)-3-(2,2,2-trifluoroethyl)-1,3-thiazolidin-4-one (corresponding to a yield of 31.2% of theory) and 58.5% (w/w) of 2-[{2-fluoro-4-methyl-5-[(2,2,2-trifluoroethyl)sulfanyl]phenyl}(2,2,2-trifluoroethyl)amino]-1,3-thiazol-4(5H)-one (corresponding to a yield of 59% of theory). The ratio of (2Z)-2-({2-fluoro-4-methyl-5-[(2,2,2-trifluoroethyl)sulfanyl]phenyl}imino)-3-(2,2,2-trifluoroethyl)-1,3-thiazolidin-4-one to 2-[{2-fluoro-4-methyl-5-[(2,2,2-trifluoroethyl)sulfanyl]phenyl}(2,2,2-trifluoroethyl)amino]-1,3-thiazol-4(5H)-one was therefore 34:66.

1. A method for preparing 2-(phenylimino)-3-alkyl-1,3-thiazolidin-4-one of formula (I) 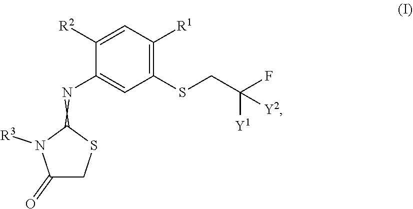
in which
Y¹ and Y² are each independently fluorine, chlorine or hydrogen,
R¹ and R² are each independently hydrogen, (C₁-C₁₂)alkyl, (C₁-C₁₂)haloalkyl, cyano, halogen or nitro, and
R³ is optionally substituted (C₆-C₁₀)aryl, (C₁-C₁₂)alkyl or (C₁-C₁₂)haloalkyl, in which the substituents are selected from halogen, (C₁-C₆)alkyl, (C₃-C₁₀)cycloalkyl, cyano, nitro, hydroxy, (C₁-C₆)alkoxy, (C₁-C₆)haloalkyl and (C₁-C₆)haloalkoxy,
Comprising reacting a 2-(phenylimino)-3H-1,3-thiazolidin-4-one of formula (VIII) 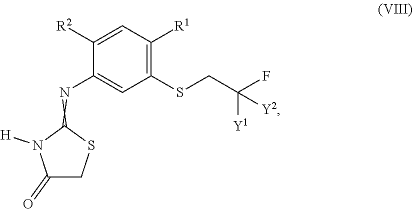
in which Y¹, Y², R¹ and R² are as defined above,
with an alkylating agent of formula (IX)
R³—Z (IX)
in which R³ is as stated above and
Z is OSO₂F
in the presence of a base and a solvent.

2. The method according to claim 1, wherein the compound of formula (VIII) is obtained from monoarylthioureas of formula (XII) 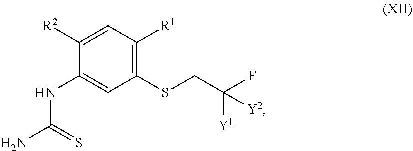
in which,
Y¹ and Y² are each independently fluorine, chlorine or hydrogen,
R¹ and R² are each independently hydrogen, (C₁-C₁₂)alkyl, (C₁-C₁₂)haloalkyl, cyano, halogen or nitro,
by reaction with a compound of formula (III) 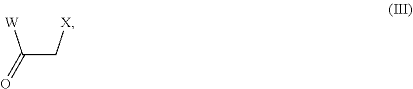
in which X is bromine, chlorine, OSO₂Me, OSO₂Ph, OSO₂(4-Me-Ph) or OSO₂CF₃ and
W is OH or an O(C₁-C₆ alkyl) radical.

3. The method according to claim 2, wherein the monoarylthiourea of formula (XII) is obtained from an aniline of the formula (IV) 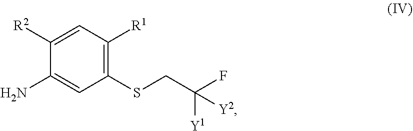
in which,
Y¹ and Y² are each independently fluorine, chlorine or hydrogen,
R¹ and R² are each independently hydrogen, (C₁-C₁₂)alkyl, (C₁-C₁₂)haloalkyl, cyano, halogen or nitro,
by reaction with an alkoxycarbonyl isothiocyanate of formula (XIII) 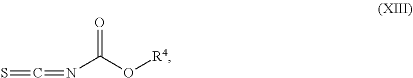
in which R⁴ is methyl, ethyl or isopropyl,
to give an alkyl (phenylcarbamothioyl)carbamate of formula (XIV) 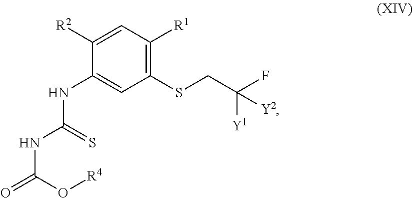
in which
Y¹ and Y² are each independently fluorine, chlorine or hydrogen,
R¹ and R² are each independently hydrogen, (C₁-C₁₂)alkyl, (C₁-C₁₂)haloalkyl, cyano, halogen or nitro,
and R⁴ is as defined above,
which is then saponified and decarboxylated under acidic or alkaline conditions.

4. The method according to claim 1, wherein the compound of formula (VIII) is obtained from a 2-halo-N-(phenyl)acetamide of formula (XV) 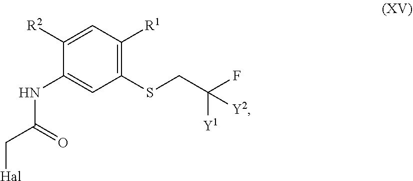
in which
Y¹ and Y² are each independently fluorine, chlorine or hydrogen,
R¹ and R² are each independently hydrogen, (C₁-C₁₂)alkyl, (C₁-C₁₂)haloalkyl, cyano, halogen or nitro and
Hal is chlorine or bromine,
by reaction with an alkali metal or ammonium rhodanide of formula (XVI)
MSCN (XVI),
in which M is Li, Na, K or NH₄.

5. The method according to claim 4, wherein the 2-halo-N-(phenyl)acetamide of the formula (XV) is obtained from an aniline of formula (IV) 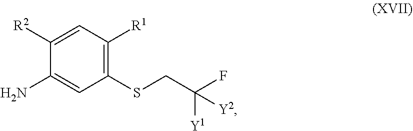
in which
Y¹ and Y² are each independently fluorine, chlorine or hydrogen,
R¹ and R² are each independently hydrogen, (C₁-C₁₂)alkyl, (C₁-C₁₂)haloalkyl, cyano, halogen or nitro,
by reaction with a haloacetyl halide of formula (XVII) 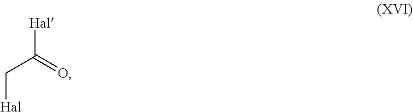
in which H is chlorine or bromine and Hal′ is chlorine or bromine.

6. The method according to claim 1, wherein
Y¹ and Y² are each independently fluorine, chlorine or hydrogen,
R¹ and R² are each independently fluorine, chlorine, (C₁-C₃)alkyl or hydrogen,
R³ is (C₁-C₆)alkyl or (C₁-C₆)haloalkyl, and
Z is OSO₂F.

7. The method according to claim 1, wherein
Y¹ and Y² are each independently fluorine or hydrogen,
R¹ and R² are each independently fluorine, chlorine, hydrogen or methyl,
R³ is (C₁-C₆)haloalkyl, and
Z is OSO₂F.

8. The method according to claim 1, wherein
Y¹ and Y² are fluorine,
R¹ and R² are each independently fluorine, hydrogen or methyl,
R³ is (C₁-C₆)fluoroalkyl, and
Z is OSO₂F.

9. The method according to claim 1, wherein
Y¹ and Y² are fluorine,
R¹ is methyl,
R² is fluorine,
R³ is CH₂CF₃, and
Z is OSO₂F.

10. The method according to claim 2, wherein X is bromine or chlorine and W is a radical O(C₁-C₆-alkyl), and optionally X is bromine or chlorine and W is a radical OCH₃ or OC₂H₅, and optionally X is bromine or chlorine and W is a radical OCH₃.

11. The method according to claim 3, wherein R⁴ is methyl or ethyl.

12. The method according to claim 4, wherein Hal is chlorine and M is Li, Na, Ka or NH₄.

13. The method according to claim 5, wherein Hal′ is chlorine.

14. The method according to claim 1, wherein the compound of formula (I) is in the form of the Z-isomer or a mixture of the E- and Z-isomers in which the proportion of the Z-isomer is greater than 50%, based on the total amount of E- and Z-isomers in the mixture.

15. The method according to claim 1, wherein the reaction of the 2-(phenylimino)-3H-1,3-thiazolidin-4-one of formula (VIII) to give the compound of formula (I) is carried out in the presence of a solvent selected from dichloromethane, acetonitrile, propionitrile, butyronitrile, ethyl acetate, butyl acetate, toluene, chlorobenzene, N,N-dimethylformamide, N,N-dimethylacetamide, N-methylpyrrolidinone, dimethyl sulfoxide, sulfolane and mixtures thereof.

16. The method according to claim 1, wherein the reaction of the 2-(phenylimino)-3H-1,3-thiazolidin-4-one of formula (VIII) to give the compound of formula (I) is carried out in the presence of a base which is selected from trimethylamine, triethylamine, tributylamine, ethyldiisopropylamine, pyridine, 2-methylpyridine, 2,3-dimethylpyridine, 2,5-dimethylpyridine, 2,6-dimethylpyridine, 2-methyl-5-ethylpyridine, quinoline, potassium methoxide, potassium ethoxide, potassium tert-butoxide, sodium methoxide, sodium ethoxide, sodium tert-butoxide, potassium acetate, sodium acetate, lithium hydroxide, potassium hydroxide, sodium hydroxide, potassium hydrogencarbonate, sodium hydrogencarbonate, potassium carbonate, sodium carbonate, caesium carbonate, calcium carbonate and magnesium carbonate.

17. The method according to claim 1, wherein said method is carried out at a temperature between −20° C. and 150° C.

18. The method according to claim 1, wherein the alkylating agent R³—Z of formula (IX) is used at a molar ratio from 0.9:1 to 2:1, based on the 2-(phenylimino)-3H-1,3-thiazolidin-4-one of formula (VIII).

19. The method according to claim 1, wherein the base is used at a molar ratio from 0.9:1 to 4:1, based on the 2-(phenylimino)-3H-1,3-thiazolidin-4-one of formula (VIII).

20. The method according to claim 1, wherein the compound (IX) is prepared in situ by reacting a compound of formula (XI)
R³—OH (XI)
in which R³ is (C₁-C₆)alkyl or (C₁-C₆)haloalkyl,
with SO₂F₂ or SO₂ClF.

21. The method according to claim 20, wherein the compound of formula (XI) is reacted with SO₂F₂.

22. The method according to claim 20, wherein the alcohol R³—OH is used at a molar ratio from 1:1 to 4:1, based on the 2-(phenylimino)-3H-1,3-thiazolidin-4-one of formula (VIII).

23. The method according to claim 20, wherein the reagent SO₂F₂ or SO₂ClF is used at a molar ratio from 1:1 to 4:1, based on the 2-(phenylimino)-3H-1,3-thiazolidin-4-one of the formula (VIII).

24. The method according to claim 20, wherein the base is used at a molar ratio from 1:1 to 4:1, based on the 2-(phenylimino)-3H-1,3-thiazolidin-4-one of formula (VIII).

25. A compound of formula (VIII) 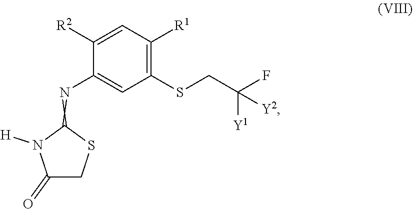
in which
Y¹ and Y² are each independently fluorine, chlorine or hydrogen,
R¹ and R² are each independently hydrogen, (C₁-C₁₂)alkyl, (C₁-C₁₂)haloalkyl, cyano, halogen or nitro.

26. A compound of formula (XII) 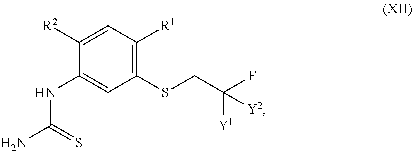
in which
Y¹ and Y² are each independently fluorine, chlorine or hydrogen,
R¹ and R² are each independently hydrogen, (C₁-C₁₂)alkyl, (C₁-C₁₂)haloalkyl, cyano, halogen or nitro.

27. A compound of formula (XIV) 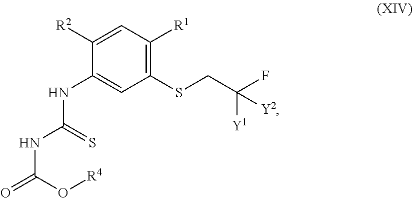
in which
Y¹ and Y² are each independently fluorine, chlorine or hydrogen,
R¹ and R² are each independently hydrogen, (C₁-C₁₂)alkyl, (C₁-C₁₂)haloalkyl, cyano, halogen or nitro
and R⁴ is methyl, ethyl or isopropyl.

28. The compound according to claim 27, in which R⁴ is methyl or ethyl.

29. A compound of formula (XV) 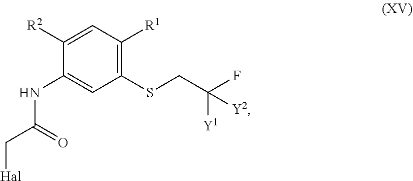
in which
Y¹ and Y² are each independently fluorine, chlorine or hydrogen,
R¹ and R² are each independently hydrogen, (C₁-C₁₂)alkyl, (C₁-C₁₂)haloalkyl, cyano, halogen or nitro;
and Hal is chlorine or bromine.

30. The compound according to claim 29, in which Hal is chlorine.

31. A compound of formula (VIII′) 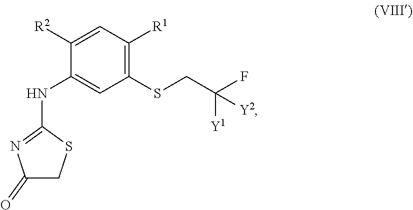
in which
Y¹ and Y² are each independently fluorine, chlorine or hydrogen,
R¹ and R² are each independently hydrogen, (C₁-C₁₂)alkyl, (C₁-C₁₂)haloalkyl, cyano, halogen or nitro.

32. A compound of formula (X) 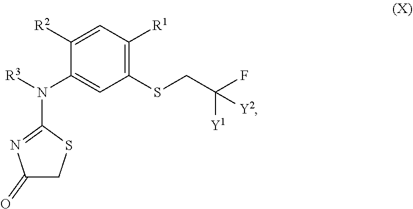
in which
Y¹ and Y² are each independently fluorine, chlorine or hydrogen,
R¹ and R² are each independently hydrogen, (C₁-C₁₂)alkyl, (C₁-C₁₂)haloalkyl, cyano, halogen or nitro,
R³ is optionally substituted (C₆-C₁₀)aryl, (C₁-C₁₂)alkyl or (C₁-C₁₂)haloalkyl, in which the substituents are selected from halogen, (C₁-C₆)alkyl, (C₃-C₁₀)cycloalkyl, cyano, nitro, hydroxy, (C₁-C₆)alkoxy, (C₁-C₆)haloalkyl and (C₁-C₆)haloalkoxy.
